(19) United States
(12) Patent Application Publication
(10) Pub. No.: US 2017/0361054 A1
(43) Pub. Date: Dec. 21, 2017

(54) MEDICAL GAS DELIVERY DEVICE

(71) Applicant: The University of Utah Research Foundation, Salt Lake City, UT (US)

(72) Inventors: Ronald Day, Cottonwood Heights, UT (US); Ryan Lechtenberg, Kearns, UT (US)

(21) Appl. No.: 15/532,984

(22) PCT Filed: Dec. 3, 2015
(86) PCT No.: PCT/US15/63781

§ 371 Date: Jun. 2, 2017

Related U.S. Application Data
(60) Provisional application No. 62/087,309, filed on Dec. 4, 2014.

(51) Int. Cl. A61M 16/12; A41D 27/28; A61M 16/16; A42B 1/04; A42C 5/04; A41D 1/04; A61M 16/08; A42B 1/00

(52) U.S. Cl. CPC A61M 16/125; A61M 16/0875; A41D 27/28; A42B 1/006; A42B 1/048; A42C 5/04; A41D 1/04; A61M 16/16; A61M 2202/0208; A41D 2200/20; A41D 2400/32; A41D 2300/324; A41D 2300/32; A61M 2210/0625; A61M 2202/0275; A61M 2202/0283; A61M 2205/59; A61M 2209/088; A61M 2210/0618; A61M 2202/0241

(57) ABSTRACT

Medical gas delivery devices are provided. A garment or another object may include a hose assembly configured to couple the garment or the object to a medical gas source. The garment or the object can be configured to deliver a medical gas to a patient. Additionally, medical gas delivery devices configured to be removably coupled to a garment or another object are provided.

### CROSS-REFERENCE TO RELATED APPLICATION

This application claims the benefit of U.S. Provisional Application No. 62/087,309, filed Dec. 4, 2014, which is hereby incorporated by reference in its entirety.

### TECHNICAL FIELD

The present disclosure relates generally to medical gas delivery devices. More specifically, the present disclosure relates to garments configured for delivery of a medical gas to a patient. Further, the present disclosure relates to medical gas delivery devices configured to be coupled to a garment.

### BRIEF DESCRIPTION OF THE DRAWINGS

The embodiments disclosed herein will become more fully apparent from the following description and appended claims, taken in conjunction with the accompanying drawings. While various aspects of the embodiments are presented in drawings, the drawings depict only typical embodiments, which will be described with additional specificity and detail through use of the accompanying drawings in which:

FIG. 1A is a perspective view of an embodiment of a medical gas delivery device in use.

FIG. 1B is a detail view of a portion of the medical gas delivery device of FIG. 1A.

FIG. 2A is a perspective view of an embodiment of a medical gas delivery tube of the medical gas delivery device of FIG. 1A.

FIG. 2B is a detail view of a portion of the medical gas delivery tube of FIG. 2A.

FIG. 3 is a front view of an embodiment of a hose assembly of the medical gas delivery device of FIG. 1A.

FIG. 4 is a side view of the medical gas delivery device of FIG. 1A.

FIG. 5A is a perspective view of another embodiment of a medical gas delivery device.

FIG. 5B is a detail view of a portion of the medical gas delivery device of FIG. 5A.

FIG. 6A is a side view of yet another embodiment of a medical gas delivery device.

FIG. 6B is a bottom perspective view of the medical gas delivery device of FIG. 6A.

FIG. 6C is a top view of the medical gas delivery device of FIG. 6A.

FIG. 7 is a perspective view of another embodiment of a medical gas delivery device.

### DETAILED DESCRIPTION

The various embodiments disclosed herein generally relate to medical gas delivery devices. In some embodiments, the medical gas delivery device comprises a garment or an article of clothing configured for delivery of a medical gas, for example, a hooded garment configured to deliver a medical gas to a patient. In some other embodiments, the medical gas delivery device is configured to be removably coupled to a garment. In yet some other embodiments, the medical gas delivery device comprises or is coupled to another object such as a blanket, pillowcase, etc.

It will be appreciated that various features are sometimes grouped together in a single embodiment, figure, or description thereof for the purpose of streamlining the disclosure. Many of these features may be used alone and/or in combination with one another.

Embodiments may be understood by reference to the drawings, wherein like parts are designated by like numerals throughout. It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail. While the various aspects of the embodiments are presented in drawings, the drawings are not necessarily drawn to scale unless specifically indicated.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device, including the devices disclosed herein. As used herein, the proximal portion of a medical gas delivery device is the portion nearest a medical gas source during use, while the distal portion is a portion at the opposite end. For example, the proximal end of a garment configured for delivery of a medical gas is defined as the end closest to the medical gas source when the garment is being worn. The distal end is the end opposite the proximal end.

The term “medical gas” refers to any medical gas including, but not limited to, oxygen, nitrous oxide, nitric oxide, anesthetic agents, medical air, inhaled breathing treatments (e.g., a bronchodilator, or a cough and/or cold relief product such as VICKS®), humidified air, and/or aromatherapy. A medical gas may be a gas (e.g., oxygen) that a practitioner desires to deliver to a patient.

Some patients, including, but not limited to, children with diseases of the heart and/or lungs (e.g., pulmonary hypertension), may not comply with the use of a face mask and/or a nasal cannula for the delivery of a medical gas (e.g., oxygen). Some methods of delivering a medical gas can comprise blowing of the medical gas toward a face of a patient; some such methods may not be effective if the patient moves his or her head away from the source of the medical gas.

A pajama, a hospital gown, a sweatshirt, a hooded article of clothing, and/or another suitable garment may be used to embody or support medical gas tubing comprising ports, holes, or openings for delivery of a medical gas to a position at or adjacent a face of a patient. In some embodiments, a medical gas source can be coupled to an inferior margin of the garment (i.e., at or adjacent a waist of the patient). In some other embodiments, a polymeric medical gas delivery tube or channel disposed within a fabric channel or covering (i.e., a mesh-like covering) may allow or permit the passage of a medical gas to one or more exit ports, holes, or openings disposed around at least a portion of a frontal opening of a hood member of a hooded garment. Such a configuration may allow or permit a medical gas to flow circumferentially through the mesh-like covering disposed at or around a face of a patient. In yet some other embodiments, another suitable object, for example, a blanket or a pillowcase, may be used to embody or support the medical gas tubing comprising ports, holes, or openings for delivery of a medical gas to a position at or adjacent a face of a patient.

In some embodiments, a medical gas delivery device of the present disclosure may allow or permit a patient to move without interrupting, or substantially interrupting, a flow of a medical gas to the patient's face. The medical gas delivery device may also be configured to limit or reduce irritation to the mouth, nose, and/or face of a patient. Additionally, the medical gas delivery device may be configured to avoid or prevent the disposition of medical gas tubing at or adjacent a neck of a patient, such that the medical gas delivery device may decrease or limit a risk that the medical gas tubing may become engaged with or wrapped around at least a portion of the neck of the patient.

A medical gas delivery device of the present disclosure may be utilized with infants, children, individuals with developmental delay, individuals who may not be cooperative or compliant with the use of a face mask and/or nasal cannula, or any other suitable individual. In certain embodiments, a medical gas delivery device may also be used with patients (i.e., patients with heart and/or lung disease) who may only need a small amount of supplemental oxygen. Additionally, a medical gas delivery device may also be used with patients having pulmonary hypertension who may only need supplemental oxygen as a pulmonary vasodilator. In some other embodiments, an individual may be anxious and/or scared when having a face mask placed over his or her mouth and/or nose (e.g., prior to or when the individual is being anesthetized). A medical gas delivery device of the present disclosure that is configured to deliver an anesthetic may be used to avoid or limit such anxiety and/or fear such that the individual may not be aware, or fully aware, of subsequent placement of a face mask during a medical procedure.

In certain embodiments, a medical gas delivery device of the present disclosure may be configured to deliver oxygen to a patient. In some embodiments, about 15% to about 45% oxygen may be sufficient. In some other embodiments, about 20% to about 35% oxygen or about 25% to about 30% oxygen may be sufficient. In yet some other embodiments, at least about 15% oxygen, at least about 20% oxygen, at least about 25% oxygen, or at least about 30% oxygen may be sufficient. Other suitable percentages of oxygen are also within the scope of this disclosure.

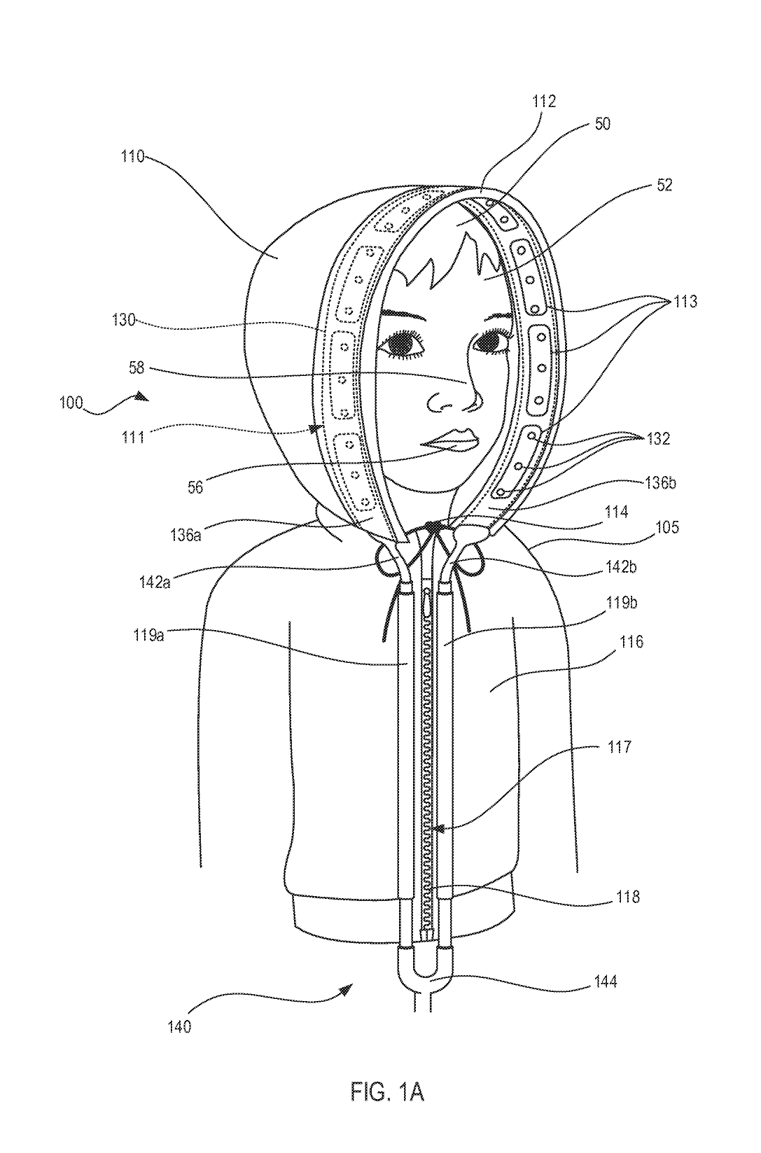

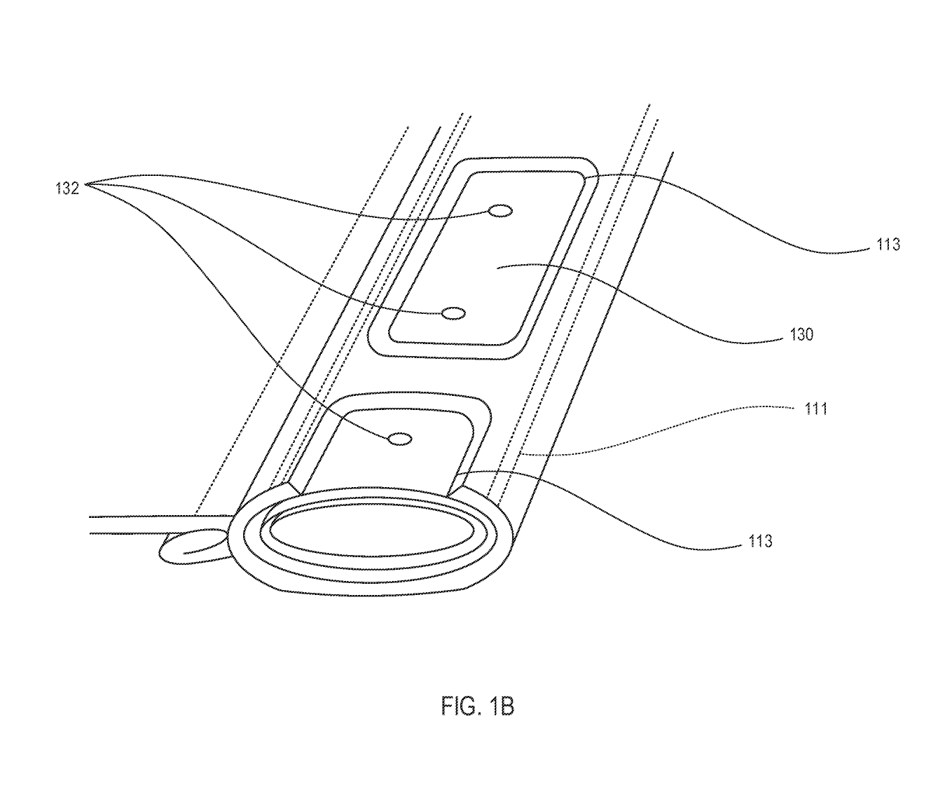

FIG. 1A is a perspective view of an embodiment of a medical gas delivery device or garment 100, wherein the medical gas delivery device 100 is in use with a patient 50. In the illustrated embodiment, the medical gas delivery device 100 comprises a hooded garment 105, wherein the hooded garment 105 may be configured to deliver a medical gas to the patient 50. The hooded garment 105 comprises a hood member 110 having a frontal or facial opening 112. The hood member 110 may be configured to removably cover at least a portion of a head 52 of the patient 50. The hood member 110, as depicted, further comprises a securement mechanism 114 (e.g., a draw string), wherein the securement mechanism 114 may be configured to at least partially secure the hood member 110 around, or to, at least a portion of the head 52 of the patient 50.

The hooded garment 105 can further comprise a torso portion 116. In some embodiments, the torso portion 116 may be configured to open and/or close at an opening region 117 (e.g., a placket). The opening region 117 may transition the hooded garment 105 between an open configuration and a closed configuration, or vice versa (e.g., when the patient 50 is putting on the hooded garment 105 and/or taking off the hooded garment 105). As illustrated in FIG. 1A, the opening region 117 comprises a coupling mechanism 118 (e.g., a zipper) such that the opening region 117 may be maintained in the closed configuration and such that the opening region 117 may also be transitioned between the closed configuration and the open configuration, or vice versa. In some other embodiments, the hooded garment 105 may not comprise an opening region; for example, the hooded garment 105 may be a pullover garment that is not configured to open and/or close at an opening region. In certain embodiments, the hooded garment 105 may be configured to open, close, and/or be coupled at the opening region 117 via at least one of a zipper, buttons, snaps, hooks, hook and loop fasteners, and/or another mechanism.

With continued reference to FIG. 1A, the hood member 110 may be coupled to a medical gas delivery tube 130. In some embodiments, the medical gas delivery tube 130 may comprise a polymeric material, a metal material, and/or another suitable material. As depicted, the medical gas delivery tube 130 is coupled to the hood member 110 at or adjacent an edge of the frontal opening 112. The medical gas delivery tube 130 of the illustrated embodiment is at least partially disposed within a hood member channel or passageway 111. The hood member channel 111 may comprise a fold or pocket formed by the fabric, or other material, from which the hooded garment 105 is made. For example, the fabric along the edge of the frontal opening 112 may be folded over and sewn, stitched, or otherwise coupled to form the hood member channel 111 for the disposition of the medical gas delivery tube 130. The hood member channel 111 may also comprise a plurality of channel openings 113 configured to allow or permit a medical gas to egress, exit, or move out of the medical gas delivery tube 130 toward at least a portion of the patient 50. In some other embodiments, the hood member channel 111 may comprise a mesh-like material and may lack the channel openings. In such embodiments, the medical gas may exit or move out of the medical gas delivery tube 130 through a mesh-like material or other porous material.

The medical gas delivery tube 130, as illustrated, may also comprise a plurality of delivery tube openings 132. As depicted, the delivery tube openings 132 are substantially evenly spaced. In certain other embodiments, the delivery tube openings 132 may be unevenly spaced. In some embodiments, a size, a shape, and/or an angle of the delivery tube openings 132 may be configured or modified such that a predetermined amount or quantity of a medical gas is directed toward a desired portion of the patient 50. The delivery tube openings 132 may also be disposed within the medical gas delivery tube 130 such that a predetermined amount or quantity of a medical gas is directed toward a desired portion of the patient 50. For example, the delivery tube openings 132 may be disposed within the medical gas delivery tube 130 such that the medical gas is directed, or at least substantially directed, toward a mouth 56 and/or a nose 58 of the patient 50. The delivery tube openings 132 can also be disposed within the medical gas delivery tube 130 such that the medical gas is directed, or at least substantially directed, toward a center portion of a face of the patient 50. In some embodiments, the medical gas delivery tube 130 and/or the delivery tube openings 132 may be configured such that the medical gas flows circumferentially around a face of the patient 50. Such a configuration may increase delivery of the medical gas to the mouth 56 and/or the nose 58 of the patient 50 in comparison to some other configurations.

In various embodiments, the medical gas delivery tube 130 may be removably coupled to the hooded garment 105. For example, the medical gas delivery tube 130 may be configured to slide into and/or out of the hood member channel 111. Such a configuration may allow or permit the hooded garment 105 and/or the medical gas delivery tube 130 to be individually cleaned, sterilized, and/or replaced.

The medical gas delivery tube 130 of FIG. 1A is disposed substantially around a circumference of a face of the patient 50. Embodiments wherein a medical gas delivery tube does not extend all the way around, or substantially around, the circumference of the face of the patient are also within the scope of this disclosure.

As depicted, the medical gas delivery device 100 further comprises a hose assembly 140 configured to couple the hooded garment 105 to one or more medical gas sources (not shown). Stated another way, the hooded garment 105 and/or the medical gas delivery tube 130 may be in fluid communication with the one or more medical gas sources via the hose assembly 140. The hose assembly 140 can be disposed along one or both edges of the opening region 117 of the hooded garment 105. In some embodiments, at least a portion of the hose assembly 140 can be coupled to the hooded garment 105 at or adjacent both of the edges of the opening region 117. As depicted, at least a portion of the hose assembly 140 is disposed within first and second torso member channels or passageways 119a, 119b disposed along at least a portion of a length of both of the edges of the opening region 117. Similar to the hood member channel 111 as described above, the first and second torso member channels 119a, 119b may comprise portions of the fabric along both of the edges of the opening region 117 that have been folded over and sewn, stitched, or otherwise coupled to form the torso member channels 119a, 119b for the disposition of at least a portion of the hose assembly 140. Such a configuration may prevent or limit one or more portions of the hose assembly 140 from becoming tangled and/or interfering with the actions or movements of the patient 50. In some other embodiments, the hose assembly 140 may be coupled to the hooded garment 105 via a different mechanism (e.g., at least one of a plurality of clips, straps, and/or tethers).

Referring again to FIG. 1A, the hose assembly 140 comprises a first secondary hose portion 142a and a second secondary hose portion 142b, wherein the first secondary hose portion 142a extends along a first edge of the opening region 117 and the second secondary hose portion 142b extends along a second edge of the opening region 117. In some embodiments, the hose assembly 140 comprising two secondary hose portions 142a, 142b can provide redundant delivery of the medical gas to the patient 50. Such a configuration may allow or permit delivery of the medical gas to the patient 50 even if one of the secondary hose portions 142a, 142b becomes pinched, occluded, or otherwise blocked. For example, if the patient 50 rolls over and pinches off or occludes the first secondary hose portion 142a, the medical gas may still be delivered to the patient 50 via the second secondary hose portion 142b.

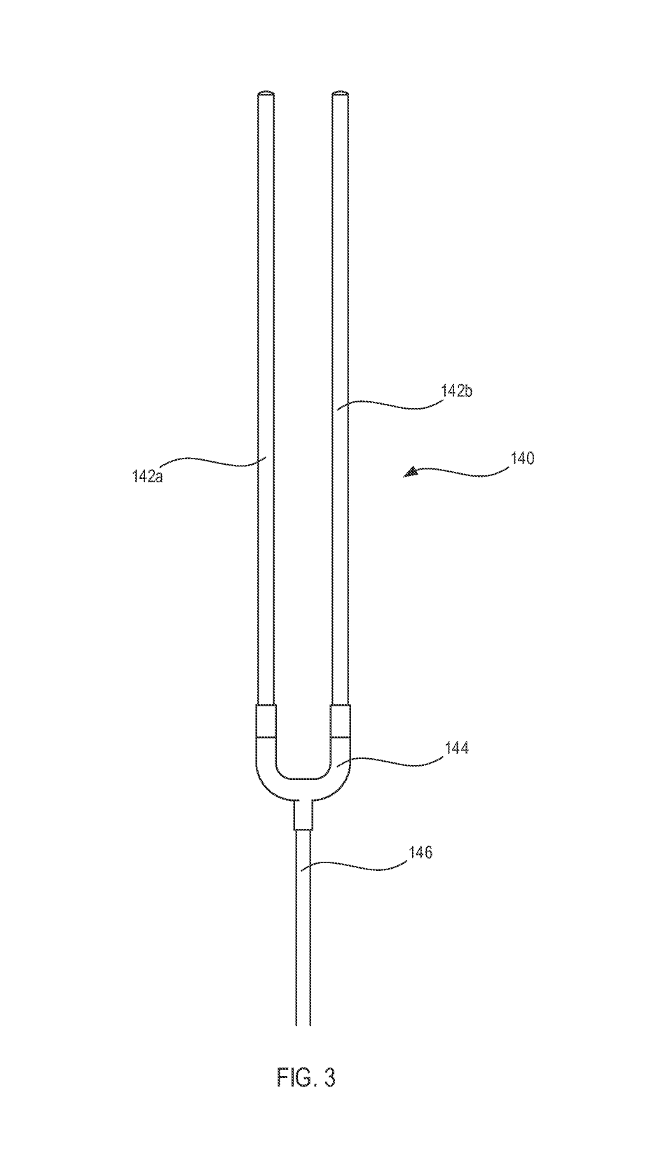

Further, the first and second secondary hose portions 142a, 142b are coupled to a bifurcated hose portion or Y-shaped hose portion 144, which may be further coupled to a primary hose portion (see primary hose portion 146 of FIG. 3), wherein the primary hose portion is disposed between the bifurcated hose portion 144 and one or more medical gas sources, and wherein the primary hose portion is in fluid communication with the one or more medical gas sources. In various embodiments, the bifurcated hose portion 144 may be disposed adjacent a neck of the patient 50. For example, a distal end of the primary hose portion may extend to a position adjacent the neck of the patient 50. Embodiments wherein the bifurcated hose portion 144 is disposed at other positions in relation to a face of a patient 50 are also within the scope of this disclosure.

In some other embodiments, the hose assembly 140 may comprise only one secondary hose portion, wherein the single secondary hose portion may be disposed along only one edge of the opening region 117 of the hooded garment 105. In yet some other embodiments, one or more components of the hose assembly 140 may be coupled to, or disposed on, other portions of the hooded garment 105. As illustrated, distal ends of the first and second secondary hose portions 142a, 142b are configured to be coupled to first and second ends 136a, 136b of the medical gas delivery tube 130, respectively. As depicted, the hose assembly 140 is coupled to both the first and second ends 136a, 136b of the medical gas delivery tube 130. In some other embodiments, however, the hose assembly 140 may be coupled to only one end of the medical gas delivery tube 130. In some embodiments, the hose assembly 140 may comprise three, four, five, or another suitable number of secondary hose portions.

FIG. 1B is a detail view of a portion of the medical gas delivery device 100 of FIG. 1A, showing a cross-section of the medical gas delivery tube 130 disposed within the hood member channel 111 of the hooded garment. In the illustrated embodiment, channel openings 113 are disposed in the material of the hood member channel 111 such that the delivery tube openings 132 in the medical gas delivery tube 130 are exposed to an exterior of the hood member and such that a medical gas may egress or exit from the medical gas delivery tube 130 via the delivery tube openings 132. As illustrated, the medical gas delivery tube 130 is flattened or ovoid. Such a configuration may limit or prevent rotation of the medical gas delivery tube 130 within the hood member channel 111 of the hooded garment such that a predetermined direction of delivery of the medical gas via the delivery tube openings 132 can be substantially maintained. In some embodiments, the medical gas delivery tube 130 may be rectangular, round, square-shaped, triangular, etc. Any other suitable shape can also be used. For example, the shape of the medical gas delivery tube 130 can be non-ovoid and/or irregular (i.e., not a traditionally defined shape).

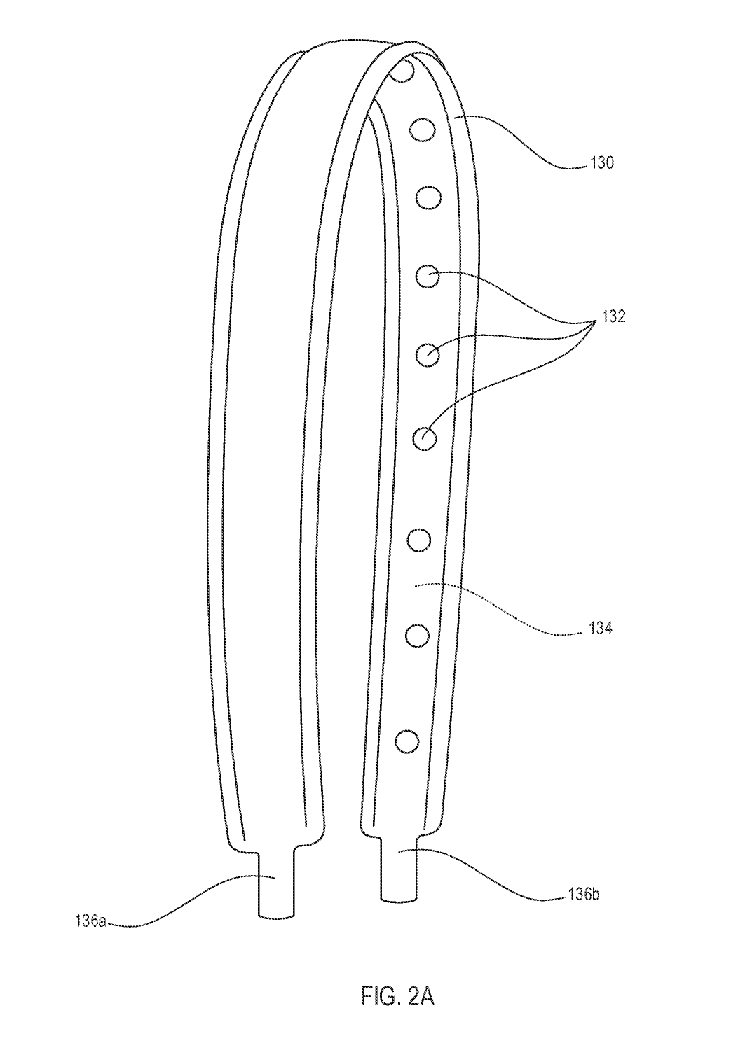

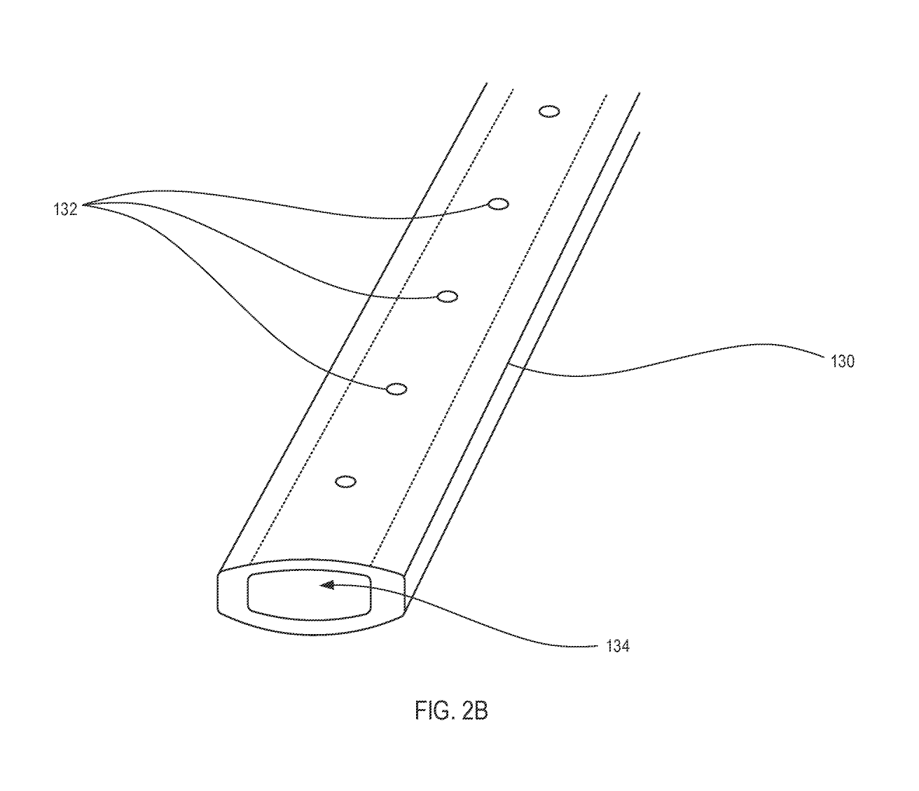

FIG. 2A is a perspective view of the medical gas delivery tube 130. As shown, the medical gas delivery tube 130 comprises a plurality of delivery tube openings 132 disposed along a length of the medical gas delivery tube 130. The delivery tube openings 132 can be disposed such that they are substantially evenly spaced and extend along substantially an entire length of the medical gas delivery tube 130. In some other embodiments, the medical gas delivery tube 130 may comprise a different number of delivery tube openings 132 from the illustrated embodiment. In certain embodiments, the delivery tube openings 132 may be arranged in a different pattern (i.e., the delivery tube openings 132 may be disposed in a zigzag pattern). The delivery tube openings 132 may also be unevenly spaced. For example, the delivery tube openings 132 may be disposed along a portion of the medical gas delivery tube 130 that is disposed most proximally to the mouth and/or the nose of the patient when the medical gas delivery tube 130 is disposed in a hooded garment and when the hooded garment is in use.

The medical gas delivery tube 130 also comprises a lumen 134 extending longitudinally through at least a portion of a length of the medical gas delivery tube 130. The lumen 134 is configured for the passage of a medical gas through at least a portion of the medical gas delivery tube 130. Additionally, as shown, the medical gas delivery tube 130 comprises a first end 136a and a second end 136b. Each of the first and second ends 136a, 136b of the medical gas delivery tube 130 may be configured to be coupled to a hose assembly (see hose assembly 140 of FIG. 1A). In some embodiments, only one of the ends 136a, 136b of the medical gas delivery tube 130 may be configured to be coupled to the hose assembly. For example, in embodiments wherein the hose assembly comprises only one secondary hose portion, the hose assembly may be coupled to only one end of the medical gas delivery tube 130. In such an embodiment, one of the first and second ends 136a, 136b of the medical gas delivery tube 130 may be sealed or may not be configured to be coupled to a hose assembly.

FIG. 2B is a detail view showing a cross-section of a portion of the medical gas delivery tube 130 of FIG. 2A. The medical gas delivery tube 130, as depicted, comprises a lumen 134 extending longitudinally through at least a portion of the medical gas delivery tube 130. The lumen 134 is configured to allow or permit the flow of a medical gas through at least a portion of the medical gas delivery tube 130. As discussed above, the medical gas delivery tube 130 further comprises a plurality of delivery tube openings 132. The delivery tube openings 132 may be configured to allow, direct, and/or permit the egress or exit of the medical gas from within the lumen 134 of the medical gas delivery tube 130 to an exterior of the medical gas delivery tube 130. In some other embodiments, the medical gas delivery tube 130 may comprise more than one lumen.

FIG. 3 is a front view of the hose assembly 140. The hose assembly 140 may comprise a primary hose portion 146, a bifurcated hose portion 144 coupled to a distal end of the primary hose portion 146, a first secondary hose portion 142a, and a second secondary hose portion 142b, wherein each of the first and second secondary hose portions 142a, 142b is coupled to a distal end of the bifurcated hose portion 144. As depicted, the components of the hose assembly 140 are discrete and are configured to be removably coupled to each other. Such a configuration may permit interchangeability in the configuration of the components of the hose assembly 140. In some other embodiments, the components of the hose assembly 140 may be unitary, wherein the components of the hose assembly 140 may be formed as a single unit.

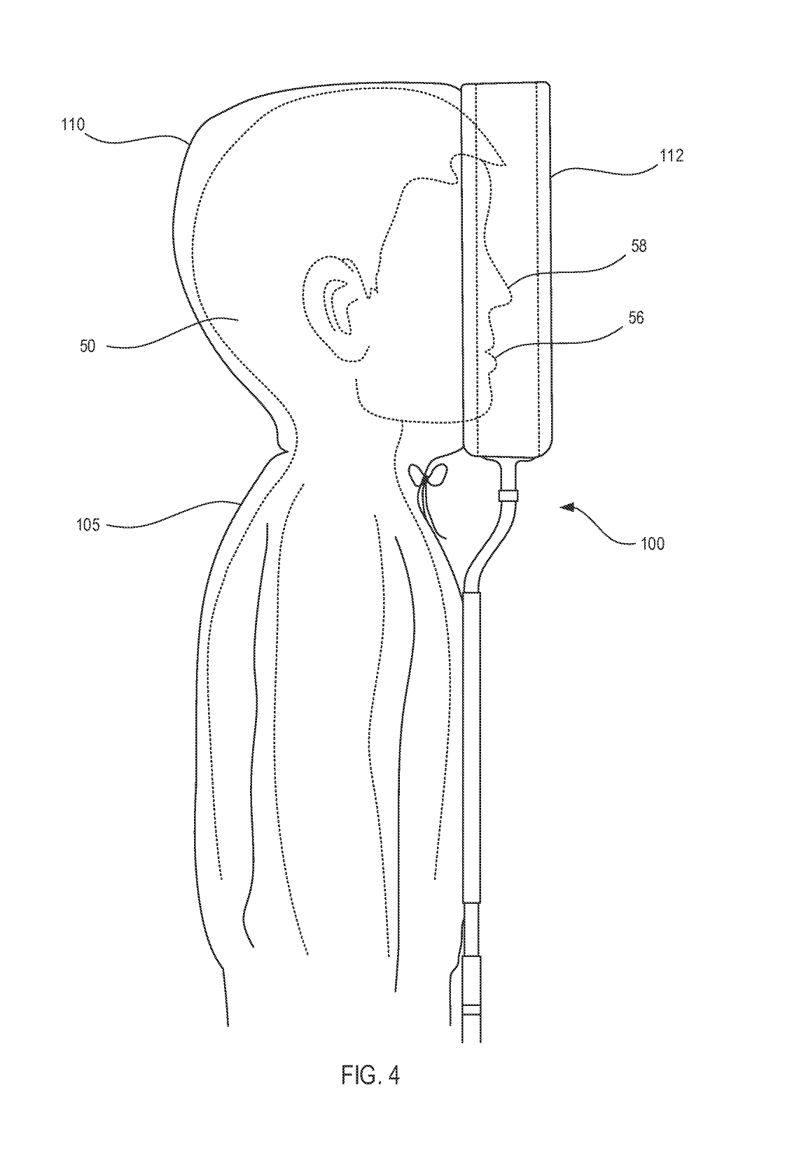

FIG. 4 is a side view of the medical gas delivery device 100 of FIG. 1A. As depicted, the distal edge of the frontal opening 112 of the hood member 110 of the hooded garment 105 is disposed at a distance away from, or distal to, the mouth 56 and the nose 58 of the patient 50 when the medical gas delivery device 100 is in use. The distance between the mouth 56 and/or the nose 58 of the patient 50 and the distal edge of the frontal opening 112 may be configured such that a predetermined amount of medical gas is delivered to the patient 50 via the medical gas delivery device 100. The distance between the mouth 56 and/or the nose 58 of the patient 50 and the distal edge of the frontal opening 112 may be changed or modified depending on the type of medical gas being delivered to the patient 50, the medical needs of the patient 50, and/or other circumstances and factors. For example, the distal edge of the frontal opening 112 may be substantially aligned with the mouth 56 and/or the nose 58 of the patient 50. In another example, the mouth 56 and/or the nose 58 of the patient 50 may be disposed at a distance away from, or distal to, the distal edge of the frontal opening 112.

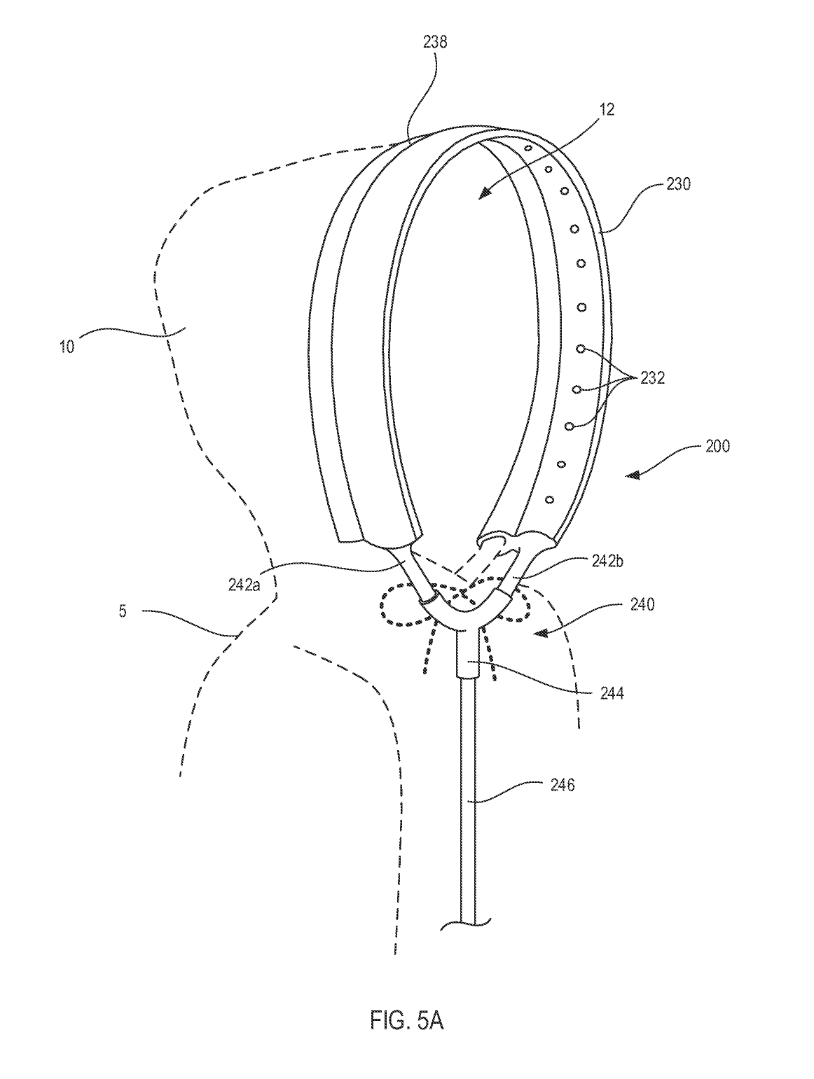

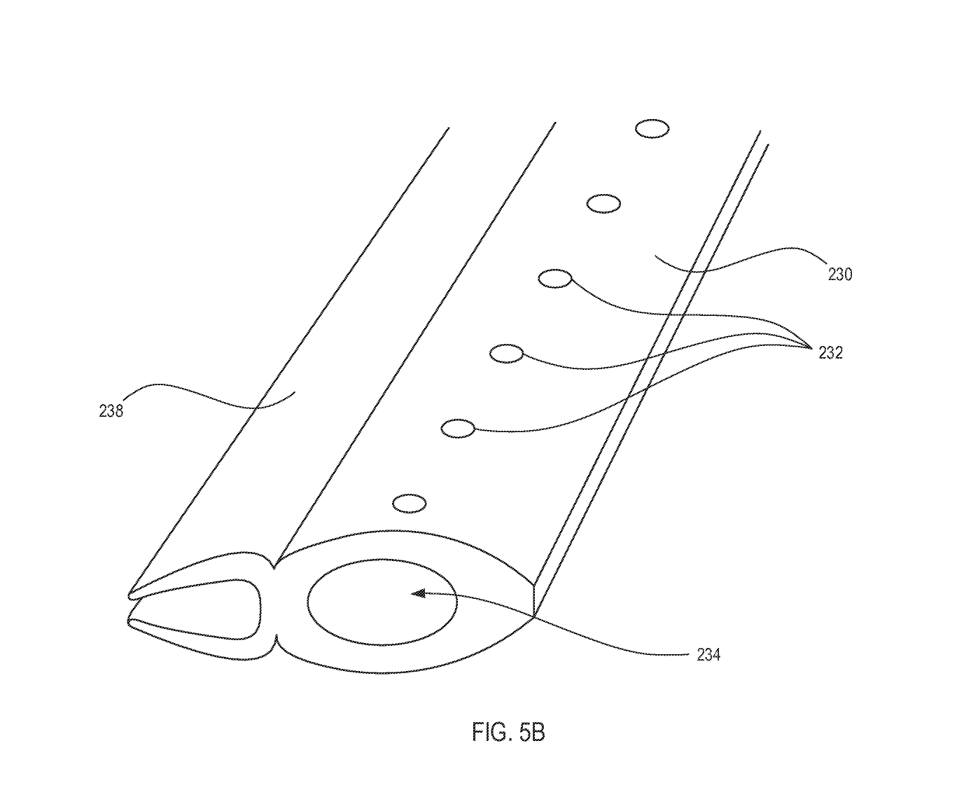

FIG. 5A illustrates another embodiment of a medical gas delivery device that can, in certain respects, resemble components of the medical gas delivery device described in connection with FIG. 1A. It will be appreciated that all the illustrated embodiments may have analogous features. Accordingly, like features are designated with like reference numerals, with the leading digits incremented to “2.” For instance, the medical gas delivery tube is designated as “130” in FIG. 1A, and an analogous medical gas delivery tube is designated as “230” in FIG. 5A. Relevant disclosure set forth above regarding similarly identified features thus may not be repeated hereafter. Moreover, specific features of the medical gas delivery device and related components shown in FIG. 1A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical gas delivery device of FIG. 5A. Any suitable combination of the features, and variations of the same, described with respect to the medical gas delivery device and components illustrated in FIG. 1A can be employed with the medical gas delivery device and components of FIG. 5A, and vice versa. This pattern of disclosure applies equally to further embodiments depicted in subsequent figures and described hereafter.

The medical gas delivery device 200 of FIG. 5A comprises the medical gas delivery tube 230. As depicted, the medical gas delivery tube 230 is configured to be removably coupled to a hood member 10 of a hooded garment 5 at or adjacent an edge of a frontal opening 12 of the hood member 10. The medical gas delivery tube 230 of the illustrated embodiment comprises a delivery tube coupling member 238. The delivery tube coupling member 238 may be configured to removably couple the medical gas delivery tube 230 to the hood member 10 of the hooded garment 5. In some embodiments, the delivery tube coupling member 238 may be configured to removably couple the medical gas delivery tube 230 to another component of the hooded garment 5, another type of garment, an object, or a patient. As depicted, the delivery tube coupling member 238 is substantially U-shaped and extends along at least a portion of a length of the medical gas delivery tube 230. The U-shaped delivery tube coupling member 238 may be formed from a substantially resilient material such that the delivery tube coupling member 238 may clip or snap onto the edge of the hood member 10 of a hooded garment 5, such that the medical gas delivery tube 230 is at least partially secured to the hood member 10. Such a configuration may allow or permit the medical gas delivery tube 230 and/or the hooded garment 5 to be separately cleaned, sterilized, and/or replaced.

The medical gas delivery tube 230, as illustrated, can also comprise a plurality of delivery tube openings 232. As depicted, the delivery tube openings 232 are substantially evenly spaced. In certain other embodiments, the delivery tube openings 232 may be unevenly spaced along a length of the medical gas delivery tube 230. In some embodiments, a size, a shape, and/or an angle of the delivery tube openings 232 may be configured or modified such that a predetermined amount or quantity of a medical gas is directed toward a desired portion of the patient. The delivery tube openings 232 may also be disposed within the medical gas delivery tube 230 such that a predetermined amount or quantity of a medical gas is directed toward a desired portion of the patient. For example, the delivery tube openings 232 may be disposed within the medical gas delivery tube 230 such that medical gas is directed, or at least substantially directed, toward a mouth and/or a nose of the patient. The delivery tube openings 232 may also be disposed within the medical gas delivery tube 230 such that medical gas is directed, or at least substantially directed, toward a center portion of a face of the patient. In some embodiments the medical gas delivery tube 230 and/or the delivery tube openings 232 may be configured such that the medical gas flows circumferentially around a face of the patient. Such a configuration may increase delivery of the medical gas to the mouth and/or the nose of the patient in comparison to some other configurations.

As depicted, the medical gas delivery device 200 further comprises a hose assembly 240 configured to couple the medical gas delivery tube 230 to one or more medical gas sources (not shown). Stated another way, the medical gas delivery tube 230 may be in fluid communication with the one or more medical gas sources via the hose assembly 240.

With continued reference to FIG. 5A, the hose assembly 240 comprises a first secondary hose portion 242a and a second secondary hose portion 242b, wherein the first secondary hose portion 242a is configured to couple the hose assembly 240 to a first end of the medical gas delivery tube 230 and wherein the second secondary hose portion 242b is configured to couple the hose assembly 240 to a second end of the medical gas delivery tube 230. Further, the first and second secondary hose portions 242a, 242b can be coupled to a bifurcated hose portion or Y-shaped hose portion 244, which is further coupled to a primary hose portion 246, wherein the primary hose portion 246 is disposed between the bifurcated hose portion 244 and one or more medical gas sources (not shown), and wherein the primary hose portion 246 is in fluid communication with the one or more medical gas sources.

In some other embodiments, the hose assembly 240 may comprise only one secondary hose portion, wherein only one end of the medical gas delivery tube 230 may be coupled to the hose assembly 240. In yet some other embodiments, one or more components of the hose assembly 140 may be coupled to, or disposed on, another portion or portions of a garment and/or a patient. In some embodiments, the hose assembly 240 may comprise three, four, five, or another suitable number of secondary hose portions.

FIG. 5B is a detail view showing a cross-section of a portion of the medical gas delivery tube 230. The medical gas delivery tube 230 comprises a lumen 234 extending longitudinally through at least a portion of the medical gas delivery tube 230. The lumen 234 is configured to allow or permit the flow of a medical gas through at least a portion of the medical gas delivery tube 230. In some embodiments, the medical gas delivery tube 230 may comprise more than one lumen. As discussed above, the medical gas delivery tube 230 further comprises a plurality of delivery tube openings 232. The delivery tube openings 232 may be configured to allow, direct, and/or permit the egress or exit of the medical gas from within the lumen 234 of the medical gas delivery tube 230 to an exterior of the medical gas delivery tube 230.

As discussed above, the medical gas delivery tube 230 can further comprise a delivery tube coupling member 238, wherein the delivery tube coupling member 238 is configured to couple the medical gas delivery tube 230 to a hooded garment, another type of garment, or a patient. As depicted, the delivery tube coupling member 238 is substantially U-shaped. In some embodiments, the delivery tube coupling member 238 may extend along substantially an entire length of the medical gas delivery tube 230. In some other embodiments, the delivery tube coupling member 238 may extend along a portion of the length of the medical gas delivery tube 230. In yet some other embodiments, the medical gas delivery tube 230 may comprise a plurality of delivery tube coupling members 238, wherein the delivery tube coupling members 238 are each discrete and are disposed a distance from one another along the length of the medical gas delivery tube 230.

The U-shaped delivery tube coupling member 238 may be formed from a resilient material such that the delivery tube coupling member 238 may be configured to clip or to snap onto the edge of a hood member of a hooded garment, and such that the medical gas delivery tube 230 can be at least partially fastened or secured to the hood member. The ends of the U-shaped portion may be disposed toward one another, as depicted, when the U-shaped portion is in a rest configuration. Upon placement of the U-shaped portion around an edge of a garment (i.e., the edge of the hood member), the ends of the U-shaped portion may remain disposed toward one another to fasten and/or secure the medical gas delivery tube 230 to the edge of the garment or to another component. Other methods of coupling the medical gas delivery tube 130 to a garment and/or a patient (e.g., via buttons, clips, hook and loop fasteners, etc.) are also within the scope of this disclosure.

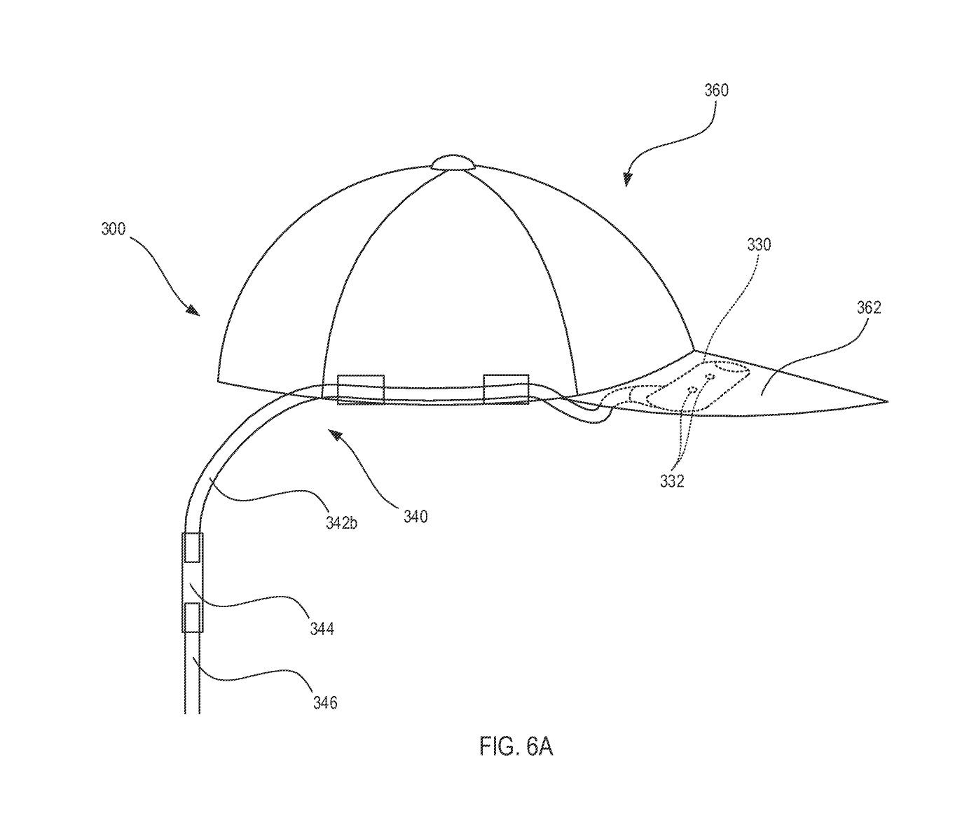

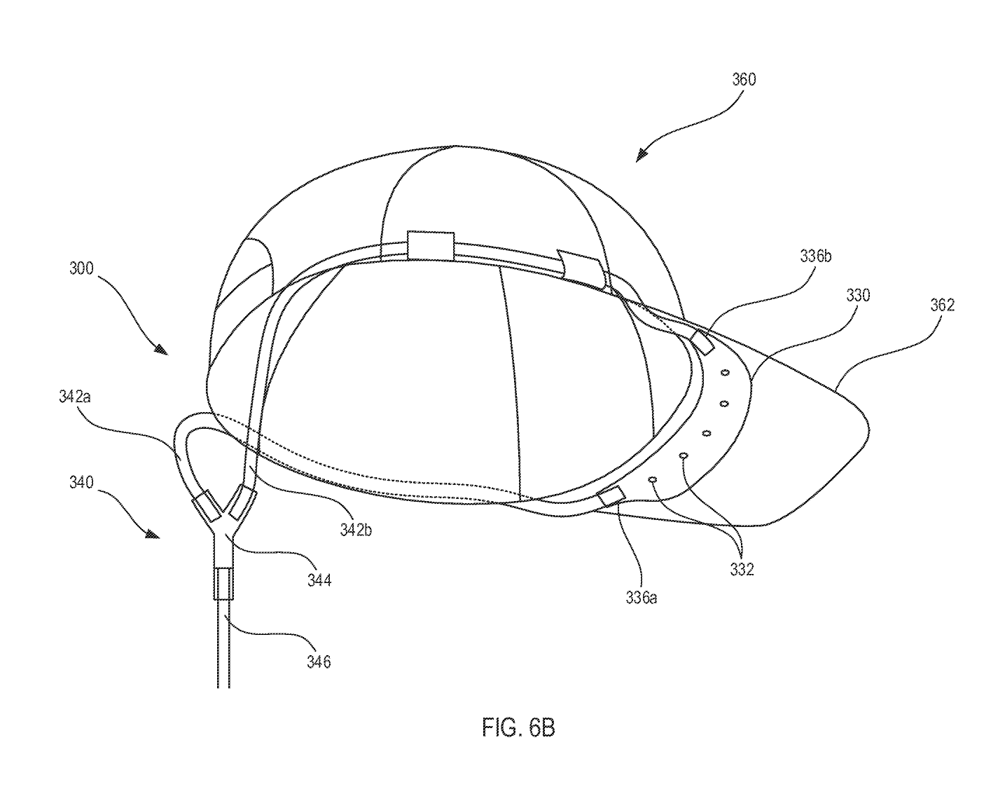

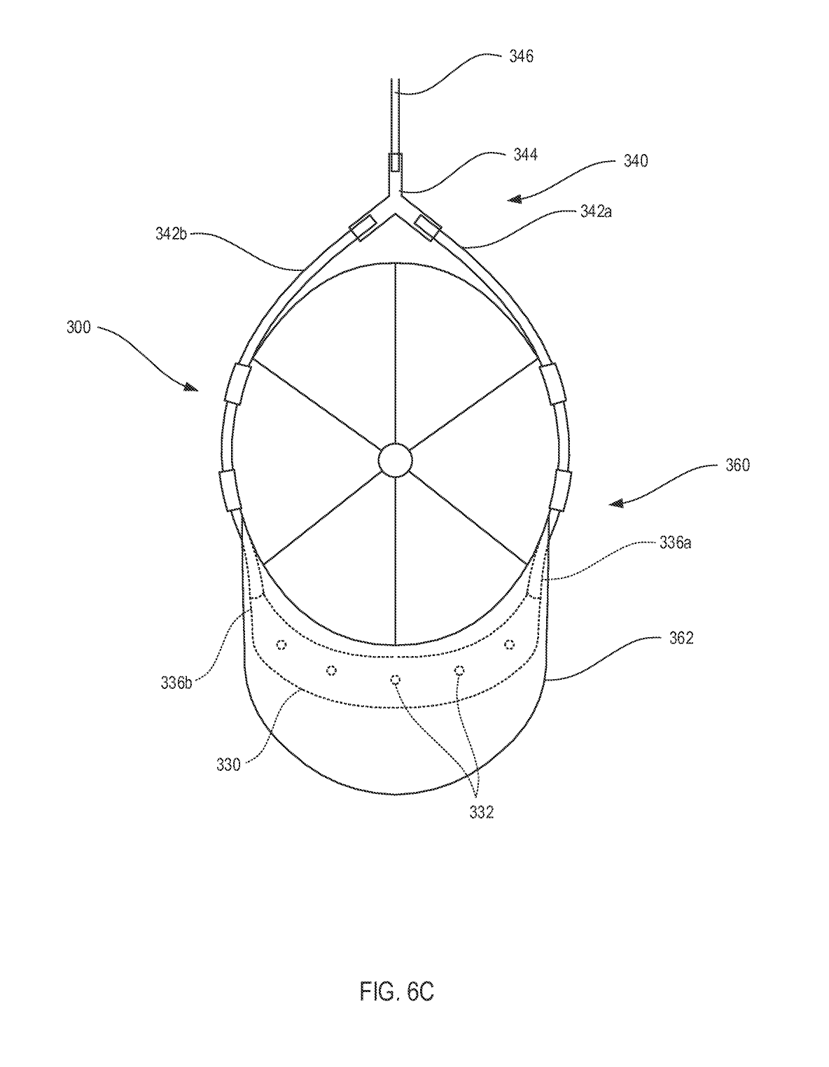

FIGS. 6A-6C depict a side view, a bottom perspective view, and a top view, respectively, of yet another embodiment of a medical gas delivery device 300. In the illustrated embodiment, the medical gas delivery device 300 comprises a hat 360 (e.g., a baseball-style cap), wherein the hat 360 may be configured to deliver medical gas to a patient. The hat 360 comprises a peak, bill, or brim 362, wherein the peak 362 can extend over and/or distal to a face of a patient.

With continued reference to FIGS. 6A-6C, the hat 360 may be coupled to a medical gas delivery tube 330. As depicted, the medical gas delivery tube 330 is coupled to a bottom surface of the peak 362. In some embodiments, the medical gas delivery tube 330 may be at least partially disposed within at least a portion of the peak 362.

The medical gas delivery tube 330, as illustrated, can also comprise a plurality of delivery tube openings 332. As depicted, the delivery tube openings 332 are substantially evenly spaced. In certain other embodiments, the delivery tube openings 332 may be unevenly spaced. The delivery tube openings 332 may also be disposed within the medical gas delivery tube 330 such that a predetermined amount or quantity of medical gas is directed toward a desired portion of a patient. For example, the delivery tube openings 332 may be disposed within the medical gas delivery tube 330 such that medical gas is directed, or at least substantially directed, toward a mouth and/or a nose of the patient.

As depicted, the medical gas delivery device 300 further comprises a hose assembly 340 configured to couple the hat 360 to one or more medical gas sources (not shown). Stated another way, the hat 360 and the medical gas delivery tube 330 may be in fluid communication with the one or more medical gas sources via the hose assembly 340. With reference to FIGS. 6B and 6C, the hose assembly 340 can be disposed along one or both lateral side bottom edges of the hat 360. In some embodiments, at least a portion of the hose assembly 340 can be coupled to the hat 360 at or adjacent the bottom edge of the hat 360 and/or a portion of the peak 362. In some embodiments, the hose assembly 340 may be coupled to the hat 360 via at least one of a plurality of clips, straps, or tethers, and/or another mechanism.

The hose assembly 340 comprises a first secondary hose portion 342a and a second secondary hose portion 342b, wherein the first secondary hose portion 342a extends along a first lateral side bottom edge of the hat 360 and the second secondary hose portion 142b extends along a second lateral side bottom edge of the hat 360. Further, the first and second secondary hose portions 342a, 342b are coupled to a bifurcated hose portion or Y-shaped hose portion 344, which may be further coupled to a primary hose portion 346, wherein the primary hose portion 346 is disposed between the bifurcated hose portion 344 and the one or more medical gas sources, and wherein the primary hose portion 346 is in fluid communication with the one or more medical gas sources.

In some other embodiments, the hose assembly 340 may comprise only one secondary hose portion, wherein the single secondary hose portion may be disposed along only one lateral side bottom edge of the hat 360. In yet other embodiments, one or more components of the hose assembly 340 may be coupled to, or disposed on, another portion or portions of the hat 360. As illustrated, distal ends of the first and second secondary hose portions 342a, 342b are configured to be coupled to first and second ends 336a, 336b of the medical gas delivery tube 330, respectively. As depicted, the hose assembly 340 is coupled to both the first and the second ends 336a, 336b of the medical gas delivery tube 330. In some other embodiments, however, the hose assembly 340 may be coupled to only one end of the medical gas delivery tube 330. In the illustrated embodiment, the hose assembly 340 comprises discrete components. In some other embodiments, the components of the hose assembly 340 may form a unitary element, wherein the components are not configured to be coupled to and decoupled from one another. As in the embodiment described above in FIGS. 5A and 5B, the medical gas delivery device 300 of FIGS. 6A-6C may analogously be configured to be removably coupled to a hat, another garment, an object, or a patient.

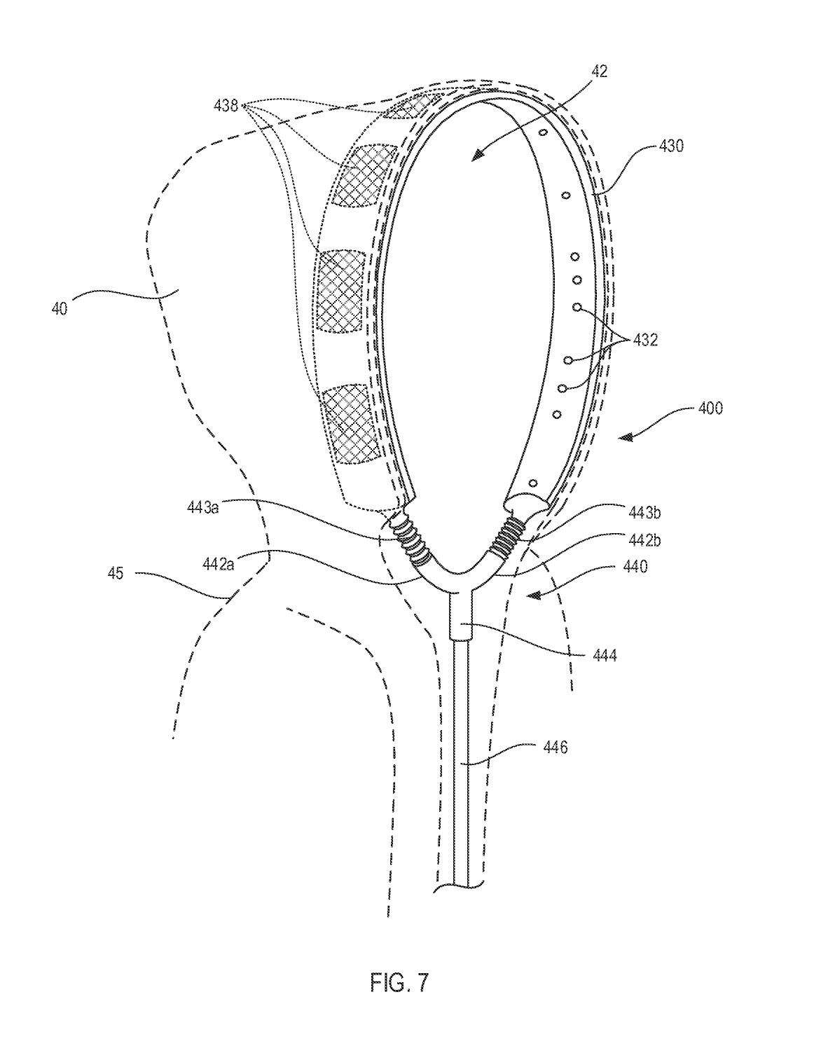

FIG. 7 illustrates yet another embodiment of a medical gas delivery device 400 comprising a medical gas delivery tube 430. As depicted, the medical gas delivery tube 430 is configured to be removably coupled to a hood member 40 of a hooded garment 45 at or adjacent an edge of a frontal opening 42 of the hood member 40. The medical gas delivery tube 430 of the illustrated embodiment comprises a plurality of delivery tube coupling members 438. The delivery tube coupling members 438 may be configured to removably couple the medical gas delivery tube 430 to the hood member 40 of the hooded garment 45. In some embodiments, the delivery tube coupling members 438 may be configured to removably couple the medical gas delivery tube 430 to another component of the hooded garment 45, another type of garment, an object, or a patient. The delivery tube coupling members 438 may comprise a hook and loop fastener, buttons, clips, snaps, and/or another suitable fastener, such that the medical gas delivery tube 430 can be at least partially secured to the hood member 40. Such a configuration may allow or permit the medical gas delivery tube 430 and the hooded garment 45 to be separately cleaned, sterilized, and/or replaced.

The medical gas delivery tube 430, as illustrated, can also comprise a plurality of delivery tube openings 432. As depicted, the delivery tube openings 432 can be unevenly spaced. As discussed above, the delivery tube openings 432 may be evenly spaced along a length of the medical gas delivery tube 430. For example, a size, a shape, and/or an angle of the delivery tube openings 432 may be configured or modified such that a predetermined amount or quantity of a medical gas is directed toward a desired portion of the patient. The delivery tube openings 432 may also be disposed within the medical gas delivery tube 430 such that a predetermined amount or quantity of a medical gas is directed toward a desired portion of the patient.

As depicted, the medical gas delivery device 400 further comprises a hose assembly 440 configured to couple the medical gas delivery tube 430 to one or more medical gas sources (not shown). As discussed above, the medical gas delivery tube 430 may be in fluid communication with the one or more medical gas sources via the hose assembly 440. With continued reference to FIG. 7, the hose assembly 440 comprises a first secondary hose portion 442a and a second secondary hose portion 442b, wherein the first secondary hose portion 442a is configured to couple the hose assembly 440 to a first end of the medical gas delivery tube 430 and wherein the second secondary hose portion 442b is configured to couple the hose assembly 440 to a second end of the medical gas delivery tube 430. Further, the first and second secondary hose portions 442a, 442b can be coupled to a bifurcated hose portion or Y-shaped hose portion 444, which is further coupled to a primary hose portion 446, wherein the primary hose portion 446 is disposed between the bifurcated hose portion 444 and the one or more medical gas sources (not shown), and wherein the primary hose portion 446 is in fluid communication with the one or more medical gas sources.

As illustrated, the first secondary hose portion 442a can include a first adjustable portion 443a, and the second secondary hose portion 442b can include a second adjustable portion 443b. Each of the first adjustable portion 443a and the second adjustable portion 443b may be configured to transition between an extended configuration or length and a contracted configuration or length. Each of the first adjustable portion 443a and the second adjustable portion 443b may also be configured to be disposed in one or more configurations or lengths between each of the extended configuration and the contracted configuration. Stated another way, lengths of each of the first adjustable portion 443a and the second adjustable portion 443b may be adjusted or transitioned such that a length of at least a portion of the medical gas delivery device 300 may be adjusted, changed, or transitioned. For example, if a patient is a newborn, the patient may have a small face, and a shorter length of at least a portion of a medical gas delivery device may be desired, and the length of one or more adjustable portions may be shortened by a user. In another example, if a patient is a teenager, the patient may have a larger face than a newborn, and a longer length of at least a portion of the medical gas delivery device may be desired, and the length of one or more adjustable portions may be lengthened by a user.

In some embodiments, the medical gas delivery device may comprise one, two, three, four, five, or more adjustable portions. In some other embodiments, the one or more adjustable portions may be disposed at or adjacent other portions of the medical gas delivery device. For example, the one or more adjustable portions may be disposed along the medical gas delivery tube, the bifurcated hose portion, the primary hose portion, etc.

### EXAMPLES

To further illustrate these embodiments, the following examples are provided. These examples are not intended to limit the scope of the claimed invention, which should be determined solely on the basis of the attached claims.

### Example 1

Breathing oxygen may increase an oxygen content of an individual. For example, the oxygen content in blood of a patient having lung disease can increase when the patient breathes an enriched amount of oxygen. In another example, blood vessels of a lung or lungs of a patient having pulmonary hypertension can relax when the patient breathes an enriched amount of oxygen. It was tested whether a 6-inch circle of oxygen tubing comprising holes spaced every 0.5 to 1.0 inches along the circle of tubing increased an amount of oxygen in a center of the circle. The amount of oxygen in the center of the circle, wherein the holes were directed toward the center of the circle, increased from 21% to 27% with an oxygen source of 5 L/minute.

### Example 2

The distribution and flow of a gas through a plurality of openings or holes in a length of tubing was tested. A circular pH indicator was prepared from coffee filter paper, with a 9-inch diameter, that had been soaked in red cabbage juice (i.e., pureed red cabbage). Upon contact with a base (e.g., ammonia), a color of the pH indicator is configured to transition from purple to green. The length of tubing was disposed along an edge of the pH indicator and then a gas comprising aerosolized ammonia and oxygen was passed through the tubing. The color of the pH indicator across substantially the entire surface of the pH indicator transitioned from purple to green, indicating contact between the pH indicator and the aerosolized ammonia. The gas that was passed through the tubing appeared to exit each of the plurality of openings disposed along the circumference of the length of tubing. The length of tubing also appeared to substantially evenly distribute the gas through each of the openings disposed along the length of the tubing.

### Example 3

A medical gas delivery tube (similar to medical gas delivery tube 130) comprising a plurality of delivery tube openings (i.e., holes) was formed. Each of the delivery tube openings extended through both an inside wall and an outside wall of the medical gas delivery tube. The medical gas delivery tube was coupled to an inside edge portion of a hood of a hooded garment, and the hooded garment was disposed on a doll of a newborn human such that the medical gas delivery tube extended around two-thirds of a circumference of a face of the doll. The doll comprised a passageway including a first opening or entrance port at a mouth of the doll and a second opening or exit port at a rear of the doll. Oxygen at various flow rates was passed through the medical gas delivery tube (1 L/minute, 2 L/minute, etc.; see Table 1). A subject inspired air at the exit port to draw the oxygen into the entrance port of the doll. A probe was placed adjacent the entrance port (“inspiring only”) or suspended about 1.5 cm above the nose of the doll (“1.5 cm above nose”), as indicated in Table 1, and the oxygen concentration was measured and recorded. The measured and recorded oxygen concentrations are shown in Table 1. The highest oxygen concentration under the “inspiring only” condition (i.e., 43%-44%) was detected at an oxygen flow rate of 6 L/minute. The highest oxygen concentration under the “1.5 cm above nose” condition (i.e., 50%-61%) was detected with an oxygen flow rate of 10 L/minute.

TABLE 1

| Flow Rate | Oxygen Conc. | Oxygen Conc. |
|---|---|---|
| (L/min.) | (inspiring only) | (1.5 cm above nose) |
| 1 | 22%-24% | 24%-29% |
| 2 | 23%-24% | 23%-29% |
| 3 | 24%-26% | 24%-35% |
| 4 | 25%-27% | 24%-39% |
| 5 | 28%-33% | 26%-44% |
| 6 | 43%-44% | 28%-48% |
| 7 | 38%-43% | 34%-54% |
| 8 | 39%-41% | 48%-58% |
| 9 | 38%-43% | 49%-61% |
| 10 | 38%-41% | 50%-61% |

### Example 4

A medical gas delivery tube (similar to medical gas delivery tube 130) comprising a plurality of delivery tube openings (i.e., holes) was formed. In contrast to the medical gas delivery tube of Example 3, each of the delivery tube openings only extended through the inside wall of the medical gas delivery tube. The medical gas delivery tube was coupled to an inside edge portion of a hood of a hooded garment, and the hooded garment was disposed on a doll of a newborn human such that the medical gas delivery tube extended around an entire circumference of a face of the doll. The doll comprised a passageway including a first opening or entrance port at a mouth of the doll and a second opening or exit port at a rear of the doll. Oxygen at various flow rates was passed through the medical gas delivery tube (1 L/minute, 2 L/minute, etc.; see Table 2). A subject inspired air at the exit port to draw the oxygen into the entrance port of the doll. A probe was placed adjacent the exit port (“exit port”), adjacent the entrance port (“inspiring only”), or suspended about 1.5 cm above the nose of the doll (“1.5 cm above nose”), as indicated in Table 2, and the oxygen concentration was measured and recorded. The measured and recorded oxygen concentrations are shown in Table 2. The highest oxygen concentration under the “inspiring only” condition (i.e., 43%-44%) was detected at an oxygen flow rate of 6 L/minute. The highest oxygen concentration under the “1.5 cm above nose” condition (i.e., 65%-68%) was detected at an oxygen flow rate of 3 L/minute.

TABLE 2

| Flow Rate | Oxygen Conc. | Oxygen Conc. | Oxygen Conc. |
|---|---|---|---|
| (L/min) | (exit port) | (inspiring only) | (1.5 cm above nose) |
| 1 | 27% | N/A | 22%-30% |
| 2 | 25% | N/A | 35%-56% |
| 3 | N/A | 24%-26% | 65%-68% |
| 4 | N/A | 25%-27% | 58%-62% |
| 5 | N/A | 28%-33% | 26%-44% |
| 6 | N/A | 43%-44% | 28%-48% |
| 7 | N/A | 38%-43% | 34%-54% |
| 8 | N/A | 39%-41% | 48%-58% |
| 9 | N/A | 38%-43% | 49%-61% |
| 10 | N/A | 38%-41% | 50%-61% |

Methods are also contemplated in connection with the devices disclosed above. Disclosure recited in connection with any device herein may be analogously applied to any method. In other words, any of the processes, steps, or functions described in connection with the devices above may be analogously incorporated into methods within the scope of this disclosure. An exemplary method relating to the devices discussed above may comprise a method of supplementing oxygen intake by a patient by disposing a medical gas delivery device of the present disclosure on the patient.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially evenly spaced” is recited with respect to a feature, it is understood that in further embodiments, the feature can have a precisely evenly spaced configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical gas delivery device, comprising:
a garment configured to deliver medical gas to a patient; and
a hose assembly coupled to a portion of the garment and configured to couple the garment to a medical gas source.

2. The medical gas delivery device of claim 1, wherein the garment is in fluid communication with the medical gas source.

3. The medical gas delivery device of claim 1, further comprising a medical gas delivery tube coupled to a hood member of the garment, the medical gas delivery tube configured to be coupled to the hose assembly, wherein the medical gas delivery tube is configured to deliver a medical gas to a mouth and/or a nose of the patient when the medical gas delivery device is in use.

4. The medical gas delivery device of claim 3, wherein the medical gas delivery tube is coupled to the hood member at or adjacent a frontal opening of the hood member, such that the medical gas delivery tube extends around a portion of a circumference of a face of the patient.

5. The medical gas delivery device of claim 3, wherein the medical gas delivery tube is disposed within a hood member channel of the hood member.

6. The medical gas delivery device of claim 5, wherein at least a portion of the hood member channel comprises a mesh material.

7. The medical gas delivery device of any one of claim 3, wherein the medical gas delivery tube comprises a plurality of delivery tube openings, wherein the delivery tube openings are disposed within the medical gas delivery tube such that the medical gas is directed toward the mouth and/or the nose of the patient when the medical gas delivery device is in use.

8. The medical gas delivery device of claim 7, wherein the plurality of delivery tube openings are configured to direct a portion of the medical gas circumferentially around a face of the patient when the medical gas delivery device is in use.

9. The medical gas delivery device of any one of claim 3, wherein the medical gas delivery tube is removably coupled to the hood member of the garment such that each of the garment and the medical gas delivery tube may be separately cleaned, sterilized, or replaced.

10. The medical gas delivery device of any one of claim 1, wherein the garment comprises:
an opening region disposed along a torso portion of the garment; and
a first edge disposed along a first side of the opening region and a second edge disposed along a second side of the opening region, wherein at least one of the first and second edges is configured to couple a portion of the hose assembly to the garment.

11. The medical gas delivery device of claim 10, further comprising a medical gas delivery tube coupled to a hood member of the garment, the medical gas delivery tube configured to be coupled to the hose assembly, wherein the medical gas delivery tube is configured to deliver a medical gas to a mouth and/or a nose of the patient when the medical gas delivery device is in use.

12. The medical gas delivery device of any one of claim 1, wherein the medical gas delivery device is configured to increase an oxygen concentration adjacent a mouth and/or a nose of the patient when the medical gas delivery device is in use.

13. The medical gas delivery device of any one of claim 1, wherein the medical gas is selected from at least one of oxygen, nitrous oxide, nitric oxide, an anesthetic agent, medical air, an inhaled breathing treatment, humidified air, or an aromatherapy.

14. A medical gas delivery device, comprising:
a hose assembly configured to be coupled to a medical gas source; and
a medical gas delivery tube configured to be coupled to the hose assembly, the medical gas delivery tube comprising a delivery tube coupling member configured to removably couple the medical gas delivery tube to a portion of a garment, wherein the medical gas delivery tube is configured to deliver a medical gas to a mouth and/or a nose of a patient when the medical gas delivery device is in use.

15. The medical gas delivery device of claim 14, wherein the portion of the garment is a hood member, and wherein the delivery tube coupling member is removably coupleable to the hood member at or adjacent a frontal opening of the hood member.

16. The medical gas delivery device of claim 14, wherein the hose assembly comprises:
a proximal end configured to couple the hose assembly to the medical gas source; and
a distal end comprising an adjustable portion, the adjustable portion configured to transition between an extended configuration and a contracted configuration, and wherein the distal end is configured to couple the hose assembly to the medical gas delivery tube.

17. The medical gas delivery device of any one of claim 14, wherein the hose assembly is configured to be coupled to each of a first end and a second end of the medical gas delivery tube such that there is redundant delivery of the medical gas to the medical gas delivery tube when the medical gas delivery device is in use.

18. The medical gas delivery device of any one of claim 14, wherein the hose assembly is removably coupleable to a portion of the garment.

19. The medical gas delivery device of any one of claim 14, wherein the medical gas is selected from at least one of oxygen, nitrous oxide, nitric oxide, an anesthetic agent, medical air, an inhaled breathing treatment, humidified air, or an aromatherapy.

20. The medical gas delivery device of any one of claim 14, wherein the medical gas delivery tube is configured to be coupled to the garment via a coupling mechanism selected from at least one of a U-shaped delivery tube coupling member, buttons, clips, snaps, or a hook and loop fastener.
